(19) United States
(12) Patent Application Publication
(10) Pub. No.: US 2004/0071968 A1
(43) Pub. Date: Apr. 15, 2004

(54) Porous compositions comprising surface modified monoliths

(76) Inventors: Agathagelos Kyrlidis, Malden, MA (US); Feng Gu, Westford, MA (US); Clayton V. McNeff, Anoka, MN (US); Peter W. Carr, St. Paul, MN (US)

Correspondence Address: Martha Finnegan, Esq., CABOT CORPORATION, 157 Concord Road, P.O. Box 7001, Billerica, MA, 01821-7001, US

(21) Appl. No.: 10/374,744

(22) Filed: Feb. 25, 2003

Related U.S. Application Data
(60) Provisional application No. 60/359,502, filed on Feb. 25, 2002.

(51) Int. Cl. B32B 9/00

(52) U.S. Cl. 428/408

(57) ABSTRACT

The present invention relates to compositions comprising surface modified carbon-clad metal oxide membranes and monoliths. In particular the invention relates to membranes and/or monoliths comprising carbon-clad metal oxide, functionalized with an organic functional group. This organic functional group, either a small molecule or a polymer, can be chosen for specific end-uses, such as selective protein binding, ion exchange, hydrophobic interaction, chiral selection to enhance separations technology.

### RELATED APPLICATIONS

This application claims the benefit of priority to U.S. Provisional Application No. 60/359,502, filed Feb. 25, 2002, the disclosure of which is incorporated herein in its entirety.

### FIELD OF THE INVENTION

The present invention relates, generally, to compositions comprising porous monoliths comprising certain carbon clad inorganic oxides having at least one organic compound attached to the surface of the monolith. These monoliths can be used as membranes or chromatography supports for molecular or biomolecular separations.

### SUMMARY OF THE INVENTION

One aspect of the present invention provides a composition comprising a porous monolith comprising carbon-clad-metal oxide. For purposes of this application, the term “metal oxide” does not include any oxide of silicon. The composition further comprises at least one organic compound attached to the carbon surface of the monolith.

Another aspect of the present invention provides a composition comprising a monolith comprising carbon-clad metal oxide, the carbon surface of the monolith being bonded to an organic group having a formula chosen from:

—Ar—(CH₂)ₘ(O(CH₂)_{y})ₙNR₂ and Ar—(CH₂)ₘ(O(CH₂)_{y})ₙN⁺R₃, wherein m, y, and n are independently chosen from zero and an integer; and R is chosen from hydrogen and alkyls;

—Ar—C(O)(O(CH₂)_{y})ₙNR₂ and Ar—C(O)(O(CH₂)_{y})ₙN⁺R₃, wherein y and n are independently chosen from zero and an integer; and R is chosen from hydrogen and alkyls;

—Ar—C(O)NH(CH₂)ₘ(O(CH₂)_{y})ₙNR₂ and Ar—C(O)NH (CH₂)ₘ(O(CH₂)_{y})ₙN⁺R₃, wherein m, y, and n are independently chosen from zero and an integer; and R is chosen from hydrogen and alkyls;

—Ar—(CH₂)ₘ(O(CH₂)_{y})ₙCOOH, wherein m, y, and n are independently chosen from zero and an integer; and R is chosen from hydrogen and alkyls;

—Ar—(CH₂)ₘ(O(CH₂)_{y})ₙSO₃H, wherein m, y, and n are independently chosen from zero and an integer; and

—Ar—(CH₂)ₘ(O(CH₂)_{y})ₙSO₃H, wherein m, y, and n are independently chosen from zero and an integer

As used above, “Ar” is an aromatic group.

In accordance with another aspect of the invention, there is provided a method for preparing such composition.

It is to be understood that both the foregoing general description and the following detailed description are exemplary and explanatory only and are not restrictive of the invention, as claimed.

### DESCRIPTION OF THE EMBODIMENTS

The present invention relates to novel porous carbon-clad metal oxide materials. In accordance with one aspect of the invention, there is provided a composition comprising a porous monolith comprising carbon-clad metal oxide and at least one organic compound attached to the carbon surface of the monolith.

In accordance with another aspect of the invention, there is provided a composition comprising a monolith comprising carbon-clad metal oxide, the carbon surface of the monolith being bonded to an organic group having a formula chosen from:

—Ar—(CH₂)ₘ(O(CH₂)_{y})ₙNR₂ and Ar—(CH₂)ₘ(O(CH₂)_{y})ₙN⁺R₃, wherein m, y, and n are independently chosen from zero and an integer; and R is chosen from hydrogen and alkyls;

—Ar—C(O)(O(CH₂)_{y})ₙNR₂ and Ar—C(O)(O(CH₂)_{y})ₙN⁺R₃, wherein y and n are independently chosen from zero and an integer; and R is chosen from hydrogen and alkyls;

—Ar—C(O)NH(CH₂)ₘ(O(CH₂)_{y})ₙNR₂ and Ar—C(O)NH (CH₂)ₘ(O(CH₂)_{y})ₙN⁺R₃, wherein m, y, and n are independently chosen from zero and an integer; and R is chosen from hydrogen and alkyls;

—Ar—(CH₂)ₘ(O(CH₂)_{y})ₙCOOH, wherein m, y, and n are independently chosen from zero and an integer; and R is chosen from hydrogen and alkyls;

—Ar—(CH₂)ₘ(O(CH₂)_{y})ₙSO₃H, wherein m, y, and n are independently chosen from zero and an integer; and

—Ar—(CH₂)ₘ(O(CH₂)_{y})ₙSO₃H, wherein m, y, and n are independently chosen from zero and an integer

As used above, “Ar” is an aromatic group.

In accordance with another aspect of the invention, there is provided a method for preparing the compositions of the invention.

In one embodiment, the carbon-clad metal oxide composition of the invention can have an open structure with a significant void volume. In one embodiment, the composition has a void volume ranging from 40% to 95% relative to the total volume of the composition. In another embodiment, the composition has a void volume ranging from 50% to 95%, such as void volume ranging from 50% to 90%, from 75% to 95%, or from 75% to 90%, relative to the total volume of the composition.

In one embodiment, the carbon-clad metal oxide composition of the invention has a surface area ranging from 10 to 300 m² per mL of the composition, such as surface areas ranging from 100 to 300 m² per mL of the composition, or from 10 to 100 m² per mL of the composition.

The carbon-clad metal oxide can comprise a variety of metal oxide materials, so long as they have sufficient structural strength to maintain the integrity of the resulting composition. “At least one” as used herein refers to “one or more,” and thus encompasses individual components as well as mixtures/combinations.

“Chosen from” as used herein also encompasses individual components as well as mixtures/combinations.

Exemplary metal oxides of the carbon-clad metal oxide include those chosen from alumina, ceria, titania, zirconia, boria, chromia, tin oxide, and nickel oxide. The oxides can also include mixtures of carbon-clad metal oxides wherein the metal oxide is alumina, ceria, titania, zirconia, boria, chromia, tin oxide, nickel oxide, and/or mixtures thereof.

The metal oxide may be produced by any technique known to those skilled in the art. For example, the production of a fumed inorganic oxide is a well-documented process involving the hydrolysis of suitable feed stock vapor, such as aluminum chloride for a fumed alumina, in a flame of hydrogen and oxygen. Molten particles of roughly spherical shape are formed in the combustion process, the diameters of which are varied through process parameters.

Precipitated metal oxides may be manufactured by utilizing conventional techniques. For example, precipitated metal oxides may be formed by the coagulation of the desired particles from an aqueous medium under the influence of high salt concentrations, acids or other coagulants. The particles can be filtered, washed, dried and separated from residues of other reaction products by conventional techniques known to those skilled in the art.

Commercially available metal oxide monolithic membranes, for example, that are available from Rhodia Orelis, Ceramem, or Exekia, can be employed.

An exemplary membrane can be a ceramic membrane element that includes multiple parallel passageways that run from one face to the opposite end face. Ceramic membranes can be formed on the internal passageway wall surfaces of a ceramic honeycomb monolith by slip casting porous coatings of ceramic particles. The coatings are dried and sintered, at elevated temperature, to bond the particles to the support and each other. An initial underlying membrane layer can have a pore size of about 0.5 μm and thickness of about 50 μm. Subsequent layers may be thinner to minimize flow resistance may contain finer particals to form finer pore sizes. Two-layer and three-layer membranes can be used for microfiltration (MF) and ultrafiltration (UF). Very fine-pored or diffusion-based membranes can be applied over a UF membrane as a final layer for evaporation or gas separations.

A monolith membrane can be made by cutting slots into one end (or both) of the monolith, and the ends of these slots can be sealed. At the opposite end of the monolith, the ends of the cells opening into the slots are sealed in a like manner. After sealing the slots/cell openings at both ends of the monolith, it is coated with membrane. The membrane element may use recrystallized silicon carbide (RSiC) monoliths as a membrane support. A full-size element (144 mm diameter×864 mm length) has about 11 m2 membrane area (2-mm passageways). The membrane module assembly can use a stainless steel housing, compressed conical stainless springs and EPDM (or Viton) boot seals. Dimensions of oxide membranes can be as follows:

| | | Length, | | |
|---|---|---|---|---|
| Products | Diameter, mm | mm | Channel, mm | Area, sq m |
| Test | 25.4 | 304 | 2 | 0.12 |
| module | | | | |
| “Beta | 67 | 864 | 2 | 2.2 |
| test” | | | | |
| module | | | | |
| Full size | 144 | 864 | 2 | 10.7 |
| module | | | | |
| | 144 | 1,500 | 2 | 18.3 |
| | 144 | 1,500 | 4 | 11.6 |
| | 200 | 1,500 | 2 | 38 |
| | 200 | 1,500 | 4 | 24 |

Exemplary membrane may include the following material and pore sizes:

| Type | Material | Nominal Pore Size |
|---|---|---|
| MF | Mixed Oxide | 0.5 μm |
| | á-alumina | 0.2 μm |
| | á-alumina | 0.1 μm |
| UF | Titania | 10 nm |

The membranes when used in microfiltration (MF), ultrafiltration (UF), or nanofiltration can also have an average pore size and composition as follows:

| | | Composition of |
|---|---|---|
| MEMBRALOX ® | Average pore size | the filtering layer |
| Microfiltration | 0.1, 0.2, 0.5, 0.8, | Alpha alumina |
| | 1.4, 2.0, 5.0 μm | |
| Ultrafiltration | 20, 50 and 100 nm | Zirconia |
| Nanofiltration | 1 and 5 kDa | Titania |

Additionally, membranes may include a permeability gradient. In such cases, the average pore sizes and the composition of the filtering layer can be as follows:

| | | | Composition of |
|---|---|---|---|
| MEMBRALOX ® | Average pore size | | the filtering layer |
| Microfiltration | 0.2, 0.5, 0.8, 1.4 | μm | Alpha alumina |
| Ultrafiltration | 100 | nm | Zirconia |

Additionally, the membrane support can be made from monolithic Al2O3 and TiO2. When the support is monolithic Al2O3 and TiO2, the membrane can have the following dimensions and composition:

| | |
|---|---|
| Support: | monolithic Al2O3 and TiO2 |
| Diameter/length: | 25 mm/1178 mm (B) |
| Number of channels: | 7 (X), 19 (W), 27 (T) |
| Diameter of channels: | 6, 3.5, 2.7 mm |
| Membrane: | ZrO2 or TiO2 |

The support can also be made from carbon. In such cases, the membranes can have the following dimensions and composition:

| | |
|---|---|
| Support: | Carbon |
| External diameter/length: | 10 mm/1200 mm |
| Number of channels: | 1 and 3 |
| Channel hydraulic diameter | 6 and 3, 6 mm |
| Membrane active coat: | ZrO2 or TiO2 |

The carbon-clad metal oxide composition is surface-modified. In one embodiment, the carbon surface of the particles is derivatized. In one embodiment, the carbon-clad metal oxide composition can be surface-modified by the attachment of an organic group to the carbon surface of the carbon-clad metal oxide monolith. Preferred processes for attaching an organic group to a carbonaceous material and examples or organic groups are described in detail in U.S. Pat. Nos. 5,554,739; 5,559,169; 5,571,311; 5,575,845; 5,630,868; 5,672,198; 5,698,016; 5,837,045; 5,922,118; 5,968,243; 6,042,643; 5,900,029; 5,955,232; 5,895,522; 5,885,335; 5,851,280; 5,803,959; 5,713,988; and 5,707,432; and International Patent Publication Nos. WO 97/47691; WO 99/23174; WO 99/31175; WO 99/51690; WO 99/63007; and WO 00/22051; all incorporated in their entirety by reference herein. These processes can be preferably used in preparing the modified carbon surface of the composition of the present invention and permit the attachment of an organic group to the carbonaceous material via a chemical reaction. As indicated above, the organic group attached to the carbonaceous material is one preferably capable of increasing the adsorption capacity and/or selectivity of the carbonaceous material and/or enhancing the resolution of solute peaks in chromatographic separations.

Once the desired application for the composition is chosen, and the particular chemical species preferably known, a particular functional group or multiple functional groups can be chosen to be attached onto the carbonaceous material in order to accomplish the selectivity needed to conduct the particular process of the selected application. One example of an application is for separation. For instance, as set forth in Garcia et al., heparin is used in the separation of lipoproteins, accordingly, heparin can be attached onto carbonaceous material in order to accomplish the desired separation. Similarly, when cationic exchange processes are needed, a sulfonic acid, for instance, can be attached on a carbonaceous material and when anionic exchanges are needed, a quaternary amine can be attached onto the carbonaceous material. Thus, with the present invention, and the knowledge possessed by one skilled in the art, separation techniques can be conducted using modified carbonaceous material to achieve the selectivity desired.

In one embodiment, the carbon surface is modified with inorganic groups, such as those used as ion exchange groups. Exemplary inorganic groups include SO₃⁻ and NH₄⁺.

As indicated above, the composition monoliths of the present invention can be used for a number of applications including, but are not limited to:

1. Chromatographic supports. These can take the form of monolithic chromatographic supports or chromatographic support beads, where each bead comprises a monolith as described herein. Grinding the solid network to a desired size can produce the beads.

2. Supports for solid phase chemistry or solid phase reagents and scavengers

3. High surface area packing for chemical reactors, for example, an immobilized enzyme reactor. In this case the porous network monolith would be formed and the active enzyme immobilized on the network by appropriate chemical linking (enzyme might be linked to monolith after monolith formation or to the fine particles prior to forming of the monolith).

4. Support for heterogeneous catalysis.

5. Microfluidics elements such as chromatography support, mixing element, catalytic support. The composition can be formed in-situ into very small channels or forms, such as microfluidic devices with dimensions of 10's to 100's of microns.

The compositions of the invention can comprise high surface area carbon-clad metal oxide materials, for maximizing convective material transport and minimizing diffusional transport limitations.

Another embodiment of the present invention provides a membrane comprising the porous composition described herein, formed into a thin film.

Moreover, the monolith can be used as a membrane that can be prepared for adsorption. Membrane adsorption can be used, for example, in protein purification. Membrane adsorption can be a competitive step to polishing chromatography (last of chromatographic steps in protein purification) in which trace impurities such as DNA, endotoxins, and host proteins, which are present in low concentration, are removed. When chromatography is used for polishing, the chromatographic column and volume of media can be relatively large (for example 100 liters). The size of the column is generally not dictated by the required adsorption capacity (impurities in low concentration), but rather the column diameter needed to handle the liquid flow rate and the column depth required to assure an even flow distribution across the column. In membrane adsorption, the chromatography column can be replaced by a polymer membrane module; the membrane can be treated to provide ion exchange or other selective retention functionality. The membrane volume can supply sufficient capacity and even flow distribution as provided by the tight membrane pore size distribution. With membrane adsorption the illustrative 100 liter chromatography column can be replaced with a 0.5-1 liter membrane module.

In one embodiment, a thin section of cross-linked carbon-clad metal oxide particle matrix can function as the substrate “membrane”. This membrane can be surfaced treated to provide the ion exchange or other surface chemistry required to effect the adsorption.

Another aspect of the present invention relates to the surface modification of a porous monolith comprising a carbon-clad metal oxide. One application for this composition is the selective separations of small molecules, enantiomers and/or biomolecules.

As indicated above, one aspect of the present invention comprises a composition comprising a porous carbon-clad metal oxide monolith . A “monolith” as used herein refers to a unitary structure, as opposed to a collection of isolated particles. In the area of separations or chromatography, monolithic chromatography supports can be advantageous over a column of packed particles. Resolution of the separation improves as the density of the packed particles increases. Usually, however, flaws or gaps occur when packing particulate supports, thereby compromising chromatographic performance. A porous carbon-clad metal oxide monolith, in contrast, bypasses the packing step by providing a single unitary support. Thus, the gaps and channels arising from a poorly packed column are eliminated.

A “carbon-clad metal oxide” as used herein refers to an metal oxide having its accessible surface partially covered or fully covered with carbon, such as by coating or depositing carbon. The carbon can be any form of carbonaceous materials that can be applied to the surface of the monolith by the skilled artisan. Preferred carbonaceous materials are capable of adsorbing an adsorbate, and includes, but are not limited to, activated carbon, carbon black, graphite, carbon formed by chemical vapor deposition, or other carbonaceous material obtained by the pyrolysis of cellulosic, fuel oil, polymeric, or other precursors. Additional examples, include but are not limited to, carbon fibers, carbon cloth, vitreous carbon, carbon aerogels, pyrolized ion exchange resins, pyrolized polymer resins, mesoporous carbon microbeads, pelleted carbon powder, nanotubes, buckyballs, silicon-treated carbon black, silica-coated carbon black, metal-treated carbon black, densified carbon black, alumina, and ceria particles, and combinations thereof or activated versions thereof.

In one embodiment, the composition comprises at least one organic compound attached to the carbon surface of the monolith comprising carbon-clad metal oxide. “Attached” as used herein, refers to adsorbing, coating, covalently bonding, ionically bonding, or any noncovalent interaction between the at least one organic compound and the carbon surface. The at least one organic compound can at least partially cover the surface, for example, fully covering or partially covering the surface, whether it be intermittent, discontinuous, patterned, or comprise a plurality of individual compounds dotting the surface. Preferred processes for attaching an organic group to a carbon material and examples of organic groups are described in detail in the patents and PCT publications which are listed above.

In one embodiment, at least one inorganic group is attached to the surface. The at least one inorganic group can, for example, be those used as ion exchange groups. Exemplary inorganic groups include S₃⁻ and NH₄⁺.

Exemplary metal oxides include ZrO₂ (zirconia), TiO₂ (titania), Al₂O₃ (alumina).

In one embodiment, the at least one organic compound is chosen from polymers, such as any of the polymers described herein. The polymer can be a thermoplastic polymeric group or a thermosetting polymeric group. The polymeric group can be a random polymer, alternating polymer, graft polymer, block polymer, star-like polymer, and/or comb-like polymer. In addition, the polymeric group can be a homopolymer, copolymer, terpolymer, and/or a polymer containing any number of different repeating units. Examples of polymeric groups include, but are not limited to, polycarbonates, polyethers, polyesters, polyacrylates, polymethacrylates, polystyrenes, polyamines, and polyolefins. Preferably, the polymeric group comprises a phenylether or bisphenyl ether and a substituted propanediyl group. For example, the polymeric group may be a polymer prepared from epoxy bisphenol-A, oligomers of epoxy bisphenol-A, or epoxy novolac. Also, the polymeric group can be attached to the pigment at multiple points along the polymer chain through proper choice of substituent groups on the repeating monomer units. The polymer can be a biopolymer, such as, but not limited to, polypeptides, and/or proteins, for example protein A, protein G, and bovine serum albumin (BSA).

The monolith surface can be coated or functionalized with other organic materials, whether monomeric, polymeric, or elastomeric. These organic materials include, but are not limited to, acrylics, alkyls, epoxies, formaldehydes, glycols, polyamides, polyesters, polyethers, polyimides, polyolefins, polyols, polysulfides, polyvinyl acetate, polyurethanes, and mixtures thereof, all of which are commercially available and are produced utilizing techniques known to those skilled in the art. The polyorganosiloxanes may include various functional groups, for example methyl, phenyl, vinyl, trifluoro, chloro and combinations thereof. Common polyorganosiloxanes include polydimethylsiloxanes, dimethylphenylmethylpolysiloxane or trifluoropropylmethylsiloxane. In addition the polyorganosiloxanes may be of a branched, cyclic, linear, or oligomeric nature and contain various functionalities including, for example: alkenyl groups; halo groups; hydroxy groups; carboxy groups; cyano groups; epoxy groups; amino groups; and combinations thereof. Examples of such resins are sold under a variety of trademarks including D.E.R.® Epoxy Resin, Drakeol® Mineral Oil, Polylite® Alkyd Resin, Epone® Epoxy Resin, and DC200® Silicone Oil.

Polymeric coatings can be applied to the carbon-clad metal oxide monoliths to change their surface properties and hydrophobicity. Coatings, such as cross-linked polybutadiene and polyethylene imine, have been known to change the retention properties of ZrO₂ spherical particles for chromatographic applications and can be used to prepare particles for reversed phase or ion exchange chromatography. Applying these coatings to inorganic oxide membranes or monoliths can lead to materials for chromatographic and separation applications.

Organic groups can be attached to the carbon surface of carbon-clad metal oxide membranes and/or monoliths to modify properties, such as hydrophobicity or hydrophilicity. In another example, the organic group can provide unique chromatographic selectivity to carbon and/or carbon coated inorganic oxide membranes and/or monoliths for chromatographic separations. Exemplary organic groups include any one or a combination of, but not limited to:

a phenyl or naphthyl group having ionic or ionizable groups;

an amino acid or derivatized amino acid, cyclodextrin, immobilized protein, polypeptide, and combinations thereof;

a fluorinated group such as a C₆F₅ group, a trifluoromethyl phenyl group, a bis-trifluorophenyl group, and combinations thereof;

—Ar—(CₙH₂ₙ₊₁)ₓ groups, wherein n is ranges from 1 to 30 and x ranges from 1 to 3;

—Ar—((CₙH₂ₙ)SO₂CH═CH₂)ₘ, wherein n ranges from 0 to 20 and m ranges from 1 to 3;

chiral ligands;

—Ar—C(CH₃)₃;

—Ar—((CₙH₂ₙ)CN)ₘ wherein n ranges from 0 to 20, and m ranges from 1 to 3;

—Ar—((CₙH₂ₙ)C(O)N(H)—CₓH₂ₓ₊₁)ₘ, wherein n ranges from 0 to 20, x ranges from 0 to 20, and m ranges from 1 to 3;

—Ar—((CₙH₂ₙ)N(H)C(O)CₓH₂ₓ₊₁l)ₘ, wherein n ranges from 0 to 20, x ranges from 0 to 20, and m ranges from 1 to 3;

—Ar—((CₙH₂ₙ)O—C(O)—N(H)—CₓH₂ₓ₊₁), wherein n ranges from 0 to 20, and m ranges from 1 to 3;

—Ar—((CₙH₂ₙ)C(O)N(H)—R)ₘ, wherein n ranges from 0 to 20, x ranges from 0 to 20, m ranges from 1 to 3, and R is an organic group capable of bonding to the nitrogen atom of amides;

—Ar—((CₙH₂ₙ)N(H)C(O)—R)ₘ, wherein n ranges from 0 to 20, x ranges from 0 to 20, m ranges from 1 to 3, and R is an organic group capable of bonding to the nitrogen atom of amides;

—Ar—((CₙH₂ₙ)O—C(O)N(H)—R)ₘ, wherein n ranges from 0 to 20, x ranges from 0 to 20, m ranges from 1 to 3, and R is an organic group capable of bonding to the nitrogen atom of amides;

optically active amino acids and derivatized optically active amino acid;

cyclodextrin attached through —Ar(CH₂)ₙ, wherein n ranges from 0 to 15.

polyethylene glycol, methoxy-terminated polyethylene glycol, resins derivatized with polyethylene glycol, and resins derivatized with methoxy-terminated polyethylene glycol;

—Ar—(CH₂)ₘ(O(CH₂)_{y})ₙNR₂ and Ar—(CH₂)ₘ(O(CH₂)_{y})ₙN⁺R₃, wherein m, y, and n are independently chosen from zero and an integer; and R is chosen from hydrogen and alkyls, such as methyl and ethyl;

—Ar—C(O)(O(CH₂)_{y})ₙNR₂ and Ar—C(O)(O(CH₂)_{y})ₙN^{+R}₃, wherein y and n are independently chosen from zero and an integer; and R is chosen from hydrogen and alkyls, such as methyl and ethyl;

—Ar—C(O)NH(CH₂)ₘ(O(CH₂)_{y})ₙNR₂ and Ar—C(O)NH (CH₂)ₘ(O(CH₂)_{y})ₙN⁺R₃, wherein m, y, and n are independently chosen from zero and an integer; and R is chosen from hydrogen and alkyls, such as methyl and ethyl;

—Ar—(CH₂)ₘ(O(CH₂)_{y})ₙCOOH, wherein m, y, and n are independently chosen from zero and an integer; and R is chosen from hydrogen and alkyls, such as methyl and ethyl;

—Ar—(CH₂)ₘ(O(CH₂)_{y})ₙSO₃H, wherein m, y, and n are independently chosen from zero and an integer;

—Ar—(CH₂)ₘ(O(CH₂)_{y})ₙSO₃H, wherein m, y, and n are independently chosen from zero and an integer;

—Ar—((CₙH₂ₙ)COOX)ₘ, wherein n ranges from 0 to 20, m ranges from 1 to 3, and X is chosen from hydrogen, cations, such as metal cations, quaternary ammonium groups, and other organic groups capable of bonding to a carboxylate;

—Ar—((CₙH₂ₙ)OH)ₘ, wherein n ranges from 0 to 20, and m ranges from 1 to 3;

—Ar—((CₙH₂ₙ)NR2)ₘ, wherein n ranges from 0 to 20, m ranges from 1 to 3, and R is chosen from hydrogen and alkyls, such as methyl or ethyl;

—Ar—((CₙH₂ₙ)NR₃X)ₘ, wherein X is an anion, and R is chosen from hydrogen and alkyls, such as methyl or ethyl;

—Ar—((CₙH₂ₙ)CHNR₃⁺COO⁻)ₘ wherein n ranges from 0 to 20, and R is chosen from hydrogen and alkyls, such as methyl or ethyl;

groups resulting from the reaction between (a) —Ar—((CₙH₂ₙ)CHNR₃⁺COO⁻)ₘ wherein n ranges from 0 to 20, and R is chosen from hydrogen and alkyls, such as methyl and ethyl, and (b) compounds containing substituents chosen from amines, hydroxyls, and carboxylic acids;

—Ar—((CₙH₂ₙ)CH═CH₂)ₘ, wherein n ranges from 0 to 20 and m ranges from 1 to 3; and

a ligand, for binding a target.

As used herein “Ar” is an aromatic group.

Examples of preferred Ar groups are described in detail in WO 02/18929, which is incorporated herein by reference.

Immobilized protein, which can be useful, for example, in the separation of racemic mixtures into their optically pure components. Cyclodextrin attached through a group —Ar(CH₂)ₙ, wherein n=0 to 15, can be useful, for example, in the separation of racemic mixtures into their optically pure components. Optically active amino acid or derivatized amino acid, can also be useful, for example, in the separation of racemic mixtures into their optically pure components.

The Lewis acid sites of ZrO₂, and TiO₂ can be used to strongly adsorb chelating chemicals, such as ethylene diamine tetramethyl phosphonic acid. The coating can be used to passivate the surfaces of these materials towards non-specific adsorption of proteins. This chemistry on particles has been shown to have some advantages in monoclonal antibody purification versus protein A supports in chromatographic separations. Applying this chemistry to membranes and/or monoliths can enable novel preparative-scale protein purification technologies. Also, attaching specific affinity ligands to the surface of carbon-clad metal oxide membranes and/or monoliths can create supports with enhanced properties for the preparative purification of proteins.

As a specific example, HPLC products can comprise the monoliths based on carbon-clad zirconia for the separation of small molecules and enantiomers. Another aspect of the invention provides a composition comprising a carbon-clad metal oxide, the surface of such material being bonded to at least one organic group having a formula chosen from:

—Ar—(CH₂)ₘ(O(CH₂)_{y})ₙNR₂ and Ar—(CH₂)ₘ(O(CH₂)_{y})ₙN⁺R₃, wherein m, y, and n are independently chosen from zero and an integer; and R is chosen from hydrogen and alkyls, such as methyl and ethyl;

—Ar—C(O)(O(CH₂)_{y})ₙNR₂ and Ar—C(O)(O(CH₂)_{y})ₙN⁺R₃, wherein y and n are independently chosen from zero and an integer; and R is chosen from hydrogen and alkyls, such as methyl and ethyl;

—Ar—C(O)NH(CH₂)ₘ(O(CH₂)_{y})ₙNR₂ and Ar—C(O)NH (CH₂)ₘ(O(CH₂)_{y})ₙN⁺R₃, wherein m, y, and n are independently chosen from zero and an integer; and R is chosen from hydrogen and alkyls, such as methyl and ethyl;

—Ar—(CH₂)ₘ(O(CH₂)_{y})ₙCOOH, wherein m, y, and n are independently chosen from zero and an integer; and R is chosen from hydrogen and alkyls, such as methyl and ethyl;

—Ar—(CH₂)ₘ(O(CH₂)_{y})ₙSO₃H, wherein m, y, and n are independently chosen from zero and an integer; and

—Ar—(CH₂)ₘ(O(CH₂)_{y})ₙSO₃H, wherein m, y, and n are independently chosen from zero and an integer.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

What is claimed is:

1. A composition comprising:
a porous monolith comprising carbon-clad metal oxide; and
at least one organic compound attached to the carbon surface of the monolith.

2. The composition according to claim 1, wherein the at least one organic compound is attached to the carbon surface by coating the surface, adsorbing to the surface, or covalently bonding to the surface.

3. The composition according to claim 1, wherein the at least one organic compound is a polymer.

4. The composition according to claim 3, wherein the polymer is chosen from polyorganosiloxanes, polycarbonates, polyethers, polyesters, polyacrylates, polymethacrylates, polystyrenes, polyamines, polyolefins, and polysaccharides.

5. The composition according to claim 1, wherein the at least one organic compound is chosen from:
phenyl and naphthyl groups having ionic or ionizable groups;
fluorinated groups;
—Ar—(CₙH₂ₙ₊₁)ₓ groups, wherein Ar is an aromatic group, n is ranges from 1 to 30 and x ranges from 1 to 3;
—Ar—((CₙH₂ₙ)SO₂CH═CH₂)ₘ, wherein Ar is an aromatic group, n ranges from 0 to 20 and m ranges from 1 to 3;
chiral ligands;
—Ar—C(CH₃)₃ wherein Ar is an aromatic group;
—Ar—((CₙH₂ₙ)CN)ₘ wherein Ar is an aromatic group, n ranges from 0 to 20, and m ranges from 1 to 3;
—Ar—((CₙH₂ₙ)C(O)N(H)—CₓH₂ₓ₊₁)ₘ, wherein Ar is an aromatic group, n ranges from 0 to 20, x ranges from 0 to 20, and m ranges from 1 to 3;
—Ar—((CₙH₂ₙ)N(H)C(O)CₓH₂ₓ₊₁)ₘ, wherein Ar is an aromatic group, n ranges from 0 to 20, x ranges from 0 to 20, and m ranges from 1 to 3;
—Ar—((CₙH₂ₙ)O—C(O)—N(H)—CₓH₂ₓ₊₁), wherein Ar is an aromatic group, n ranges from 0 to 20, and m ranges from 1 to 3;
—Ar—((CₙH₂ₙ)C(O)N(H)—R)ₘ, wherein Ar is an aromatic group, n ranges from 0 to 20, x ranges from 0 to 20, m ranges from 1 to 3, and R is an organic group;
—Ar—((CₙH₂ₙ)N(H)C(O)—R)ₘ, wherein Ar is an aromatic group, n ranges from 0 to 20, x ranges from 0 to 20, m ranges from 1 to 3, and R is an organic group;
—Ar—((CₙH₂ₙ)O—C(O)N(H)—R)ₘ, wherein Ar is an aromatic group, n ranges from 0 to 20, x ranges from 0 to 20, m ranges from 1 to 3, and R is an organic group;
optically active amino acids and derivatized optically active amino acid; and
cyclodextrin attached through —Ar(CH₂)ₙ, wherein Ar is an aromatic group, n ranges from 0 to 15.

6. The composition according to claim 1, wherein the at least one organic compound is chosen from amino acids, derivatized amino acids, cyclodextrin, proteins, and polypeptides.

7. The composition according to claim 1, wherein the at least one organic compound is chosen from polyethylene glycol, methoxy-terminated polyethylene glycol, resins derivatized with polyethylene glycol, and resins derivatized with methoxy-terminated polyethylene glycol.

8. The composition according to claim 1, wherein the at least one organic compound is a group having a formula chosen from —Ar—(CH₂)ₘ(O(CH₂)_{y})ₙNR₂ and Ar—(CH₂)ₘ(O(CH₂)_{y})ₙN^{+R}₃, wherein Ar is an aromatic group; m, y, and n are independently chosen from zero and an integer; and R is chosen from hydrogen and alkyls.

9. The composition according to claim 1, wherein the at least one organic compound is a group having a formula chosen from —Ar—C(O)(O(CH₂)_{y})ₙNR₂ and Ar—C(O)(O(CH₂)_{y})ₙN⁺R₃, wherein Ar is an aromatic group; y and n are independently chosen from zero and an integer; and R is chosen from hydrogen and alkyls.

10. The composition according to claim 1, wherein the at least one organic compound is a group having a formula chosen from —Ar—C(O)NH(CH₂)ₘ(O(CH₂)_{y})ₙNR₂ and Ar—C(O)NH(CH₂)ₘ(O(CH₂)_{y})ₙN⁺R₃, wherein Ar is an aromatic group; m, y, and n are independently chosen from zero and an integer; and R is chosen from hydrogen and alkyls.

11. The composition according to claim 1, wherein the at least one organic compound has a formula —Ar—(CH₂)ₘ(O(CH₂)_{y})ₙCOOH, wherein Ar is an aromatic group; m, y, and n are independently chosen from zero and an integer; and R is chosen from hydrogen and alkyls.

12. The composition according to claim 1, wherein the at least one organic compound has a formula —Ar—(CH₂)ₘ(O(CH₂)_{y})ₙSO₃H, wherein Ar is an aromatic group, and m, y, and n are independently chosen from zero and an integer.

13. The composition according to claim 1, wherein the at least one organic compound has a formula —Ar—(CH₂)ₘ(O(CH₂)_{y})ₙSO₃H, wherein Ar is an aromatic group, and m, y, and n are independently chosen from zero and an integer.

14. The composition according to claim 1, wherein the at least one organic compound has a formula —Ar—((CₙH₂ₙ)COOX)ₘ, wherein Ar is an aromatic group, n ranges from 0 to 20, m ranges from 1 to 3, and X is chosen from hydrogen, cations, and organic groups.

15. The composition according to claim 1, wherein the at least one organic compound has a formula —Ar—((CₙH₂ₙ)OH)ₘ, wherein Ar is an aromatic group, n ranges from 0 to 20, and m ranges from 1 to 3.

16. The composition according to claim 1, wherein the at least one organic compound has a formula —Ar—((CₙH₂ₙ)NR₂)ₘ, wherein Ar is an aromatic group, n ranges from 0 to 20, m ranges from 1 to 3, and R is chosen from hydrogen and alkyls.

17. The composition according to claim 1, wherein the at least one organic compound has a formula —Ar—((CₙH₂ₙ)NR₃X)ₘ, wherein X is an anion, Ar is an aromatic group, and R is chosen from hydrogen and alkyls.

18. The composition according to claim 1, wherein the at least one organic compound has a formula —Ar—((CₙH₂ₙ)CHNR₃⁺COO⁻)ₘ wherein Ar is an aromatic group, n ranges from 0 to 20, and R is chosen from hydrogen and alkyls.

19. The composition according to claim 1, wherein the at least one organic compound is chosen from groups resulting from the reaction between (a) —Ar—((CₙH₂ₙ)CHNR₃⁺COO⁻)ₘ wherein Ar is an aromatic group, n ranges from 0 to 20, and R is chosen from hydrogen and alkyls, and (b) compounds containing substituents chosen from amines, hydroxyls, and carboxylic acids,

20. The composition according to claim 1, wherein the at least one organic compound has a formula —Ar—((CₙH₂ₙ)CH═CH₂)ₘ, wherein n ranges from 0 to 20 and m ranges from 1 to 3.

21. The composition according to claim 1, wherein the at least one organic compound is a ligand, for binding a target.

22. A chromatography column comprising the composition according to claim 1.

23. A membrane comprising the composition according to claim 1.

24. The membrane according to claim 23, wherein the membrane is a film.

25. A composition comprising a material comprising carbon-clad metal oxide, the carbon surface of the material being bonded to an organic group having a formula chosen from:
—Ar—(CH₂)ₘ(O(CH₂)_{y})ₙNR₂ and Ar—(CH₂)ₘ(O(CH₂)_{y})ₙN⁺R₃, wherein Ar is an aromatic group; m, y, and n are independently chosen from zero and an integer; and R is chosen from hydrogen and alkyls;
—Ar—C(O)(O(CH₂)_{y})ₙNR₂ and Ar—C(O)(O(CH₂)_{y})ₙN⁺R₃, wherein Ar is an aromatic group; y and n are independently chosen from zero and an integer; and R is chosen from hydrogen and alkyls;
—Ar—C(O)NH(CH₂)ₘ(O(CH₂)_{y})ₙNR₂ and Ar—C(O)NH(CH₂)ₘ(O(CH₂)_{y})ₙN^{+R}₃, wherein Ar is an aromatic group; m, y, and n are independently chosen from zero and an integer; and R is chosen from hydrogen and alkyls;
—Ar—(CH₂)ₘ(O(CH₂)_{y})ₙCOOH, wherein Ar is an aromatic group; m, y, and n are independently chosen from zero and an integer; and R is chosen from hydrogen and alkyls;
—Ar—(CH₂)ₘ(O(CH₂)_{y})ₙSO₃H, wherein Ar is an aromatic group, and m, y, and n are independently chosen from zero and an integer; and
—Ar—(CH₂)ₘ(O(CH₂)_{y})ₙSO₃H, wherein Ar is an aromatic group, and m, y, and n are independently chosen from zero and an integer.
